(19) United States
(12) Patent Application Publication
(10) Pub. No.: US 2019/0194588 A1
(43) Pub. Date: Jun. 27, 2019

(54) PARTITIONING DEVICE FOR CREATING A COMPETITIVE ASSAY PLATFORM

(71) Applicant: The Board of Trustees of the University of Alabama, Tuscaloosa, AL (US)

(72) Inventors: Shreyas S. Rao, Tuscaloosa, AL (US); Yuping Bao, Tuscaloosa, AL (US); Akshay A. Narkhede, Tuscaloosa, AL (US); Jennifer Sherwood, Northport, AL (US); Doug Cannon, Gordo, AL (US)

(21) Appl. No.: 16/223,933

(22) Filed: Dec. 18, 2018

Related U.S. Application Data
(60) Provisional application No. 62/599,890, filed on Dec. 18, 2017.

(51) Int. Cl. C12M 1/00; C12M 1/32; C12M 1/12

(52) U.S. Cl. CPC C12M 23/34; C12M 25/02; C12M 23/12

(57) ABSTRACT

Various implementations include a competitive assay platform with a partitioning device disposed in a well. The well has a bottom surface, a central axis, and a side surface. The partitioning device includes a first wall portion, a second wall portion, and a third wall portion. Each wall portion has an upper edge, a lower edge, a first side edge, a second side edge, and first and second divider surfaces. The lower edge of the first wall portion, second wall portion, and third wall portion of the partitioning device disposed on the bottom surface of the well, and the first side edges of the first, second, and third wall portions are disposed against the side surface of the well, defining first, second, and third compartments.

### CROSS REFERENCE TO RELATED APPLICATIONS

This application claims the benefit of U.S. Provisional Patent Application No. 62/599,890, filed Dec. 18, 2017, the content of which is incorporated herein by reference in its entirety.

### STATEMENT REGARDING FEDERALLY SPONSORED RESEARCH

This invention was made with government support under Grant No. DMR1149931 awarded by the National Science Foundation. The government has certain rights in the invention.

### BACKGROUND

Several previous in vitro co-culture systems have been developed to study interaction and selective uptake of substances such as nanoparticles with various cell types. However, these systems make it relatively difficult to incorporate more than two cell types, and additional labeling techniques must be employed to distinguish various cell types. Thus, there is a need in the art for improved systems and methods for studying selective uptake of substances by cell cultures.

### SUMMARY

Various implementations of competitive assay platforms can be used to study selective uptake of substances (e.g., nanoparticles, drugs, heavy metals, chemicals) in three dimensional (3D) cultures. In some implementations, the platform includes a partitioning device that divides a volume of a well into multiple compartments for receiving different 3D cultures. In certain implementations, the partitioning device is separately formed from and fits into a well of a standard cell culture well plate or other type of well structure. In other implementations, the partitioning device is integrally formed with the well surfaces (e.g., a bottom surface and side surface(s) of the well are integrally formed in one molding process with the partitioning device). In some implementations, the partitioning device is made of biocompatible polymers (e.g., poly(lactic acid) (PLA)). And, in some implementations, the partitioning device may be made using additive manufacturing. A substance, such as nanoparticles, may be introduced into the well to evaluate selective uptake of the substance by the 3D cultures. Because of its broad utility, this platform can be easily adapted with standard culture substrates in a range of tissue-like environments and, thus, can be readily utilized by laboratories studying nanoparticle-cellular interactions.

In addition, this technology could be broadly applied for fundamental studies of preferential cellular decisions in physiologically relevant 3D environments.

Various implementations include a partitioning device for creating at least three separate three-dimensional cultures for studying selective uptake of a substance in the cultures. The partitioning device is disposable in a well that is defined by a bottom surface through which a central axis of the well extends and at least one side surface that extends axially from the bottom surface. The partitioning device includes a first wall, a second wall, and a third wall.

The first wall portion has an upper edge, a lower edge, a first side edge, a second side edge, and first and second divider surfaces. The upper and lower edges are opposite and spaced apart from each other, the first and second side edges are opposite and spaced apart from each other, and the first and second divider surfaces are opposite and spaced apart from each other and extend between the upper and lower edges and the first and second side surfaces.

The second wall portion has an upper edge, a lower edge, a first side edge, a second side edge, and first and second divider surfaces. The upper and lower edges of the second wall portion are opposite and spaced apart from each other, the first and second side edges of the second wall portion are opposite and spaced apart from each other, and the first and second divider surfaces of the second wall portion are opposite and spaced apart from each other and extend between the upper and lower edges and the first and second side surfaces of the second wall portion.

The third wall portion has an upper edge, a lower edge, a first side edge, a second side edge, and first and second divider surfaces. The upper and lower edges of the third wall portion are opposite and spaced apart from each other, the first and second side edges of the third wall portion are opposite and spaced apart from each other, and the first and second divider surfaces of the third wall portion are opposite and spaced apart from each other and extend between the upper and lower edges and the first and second side surfaces of the third wall portion.

The lower edge of the first, second, and third wall portions are disposable on a bottom surface of a well. The first side edges of the first, second, and third wall portions are disposable against at least one side surface of the well. The first and second divider surfaces of the first, second, and third wall portions at least partially define first, second, and third compartments.

In some implementations, the second side edges of the first, second, and third wall portions intersect each other.

In some implementations, the first side edges of the first, second, and third wall portions are coupled to each other by a coupling wall, the coupling wall being disposable against at least one side surface of the well.

In some implementations, the partitioning device further includes a center wall having a perimeter forming a closed shape, and the center wall is sealingly disposable between the second side edges of the first, second, and third wall portions.

In some implementations, each of the first wall portion, the second wall portion, and the third wall portion are planar.

In some implementations, the first wall portion, the second wall portion, and/or the third wall portion are integrally formed.

In some implementations, the partitioning device further includes a fourth wall portion. The fourth wall portion has an upper edge, a lower edge, a first side edge, a second side edge, and first and second divider surfaces. The upper and lower edges of the fourth wall portion are opposite and spaced apart from each other, the first and second side edges of the fourth wall portion are opposite and spaced apart from each other, and the first and second divider surfaces of the fourth wall portion are opposite and spaced apart from each other and extend between the upper and lower edges and the first and second side surfaces of the fourth wall portion. The first side edge of the fourth wall portion is disposable against at least one side surface of the well, and first and second divider surfaces of the first, second, third, and fourth wall portions at least partially define the first, second, and third compartments and a fourth compartment.

In some implementations, the partitioning device comprises a biocompatible material. In some implementations, the biocompatible material comprises a polymer. In some implementations, the biocompatible material comprises polylactic acid. In some implementations, the biocompatible material comprises polycaprolactone.

In some implementations, the partitioning device comprises a non-biocompatible material.

In some implementations, the compartments have equal volumes.

In some implementations, the wall portions of the partitioning device are solid.

In some implementations, the wall portions of the partitioning device prevent flow of a pourable culture substrate between the compartments. In some implementations, the wall portions of the partitioning device comprise one or more openings through which uptake materials flow between the compartments. In some implementations, the wall portions of the partitioning device comprise a membrane through which uptake materials flow between the first and second compartments.

In some implementations, the partitioning device is created using additive manufacturing.

In some implementations, the partitioning device further includes at least one well. The at least one well is defined by a bottom surface through which a central axis extends and at least one side surface extending axially from the bottom surface. The first, second, and third wall portions are disposed within the well. In some implementations, the lower edges of the first, second, and third wall portions are disposed against the bottom surface of the well, and the first side edges of the first, second, and third wall portions are disposed in contact with at least one side surface of the well.

In various other implementations, a competitive assay system includes at least one well and a partitioning device. The at least one well is defined by a bottom surface through which a central axis extends and at least one side surface extending axially from the bottom surface.

The partitioning device is disposed within the at least one well. The partitioning device includes a first wall, a second wall, and a third wall. The first wall portion has an upper edge, a lower edge, a first side edge, a second side edge, and first and second divider surfaces. The upper and lower edges are opposite and spaced apart from each other, the first and second side edges are opposite and spaced apart from each other, and the first and second divider surfaces are opposite and spaced apart from each other and extend between the upper and lower edges and the first and second side surfaces.

The second wall portion has an upper edge, a lower edge, a first side edge, a second side edge, and first and second divider surfaces. The upper and lower edges of the second wall portion are opposite and spaced apart from each other, the first and second side edges of the second wall portion are opposite and spaced apart from each other, and the first and second divider surfaces of the second wall portion are opposite and spaced apart from each other and extend between the upper and lower edges and the first and second side surfaces of the second wall portion.

The third wall portion has an upper edge, a lower edge, a first side edge, a second side edge, and first and second divider surfaces. The upper and lower edges of the third wall portion are opposite and spaced apart from each other, the first and second side edges of the third wall portion are opposite and spaced apart from each other, and the first and second divider surfaces of the third wall portion are opposite and spaced apart from each other and extend between the upper and lower edges and the first and second side surfaces of the third wall portion.

The lower edge of the first, second, and third wall portions are disposed against the bottom surface of a well. The first side edges of the first, second, and third wall portions are disposed in contact with at least one side surface of the well. The first and second divider surfaces of the first, second, and third wall portions at least partially define first, second, and third compartments.

In some implementations, the second side edges of the first, second, and third wall portions intersect each other.

In some implementations, the first side edges of the first, second, and third wall portions are coupled to each other by a coupling wall, the coupling wall being disposable against at least one side surface of the well.

In some implementations, the partitioning device further includes a center wall having a perimeter forming a closed shape, and the center wall is sealingly disposable between the second side edges of the first, second, and third wall portions.

In some implementations, the partitioning device is separately formed from the side surface and bottom surface of the well such that the partitioning device is removably disposable within the well.

In some implementations, each of the first, second, and third wall portions of the partitioning device are planar.

In some implementations, the partitioning device further includes a fourth wall portion. The fourth wall portion has a lower edge and an upper edge, first and second side edges, and first and second divider surfaces that are opposite and spaced apart from each other. The first side edge of the fourth wall portion is disposed in contact with at least one side surface of the well, and the divider surfaces of the wall portions define the first, second, and third compartments and a fourth compartment.

In some implementations, the partitioning device comprises a biocompatible material. In some implementations, the biocompatible material comprises a polymer. In some implementations, the biocompatible material comprises polylactic acid. In some implementations, the biocompatible material comprises polycaprolactone.

In some implementations, the partitioning device comprises a non-biocompatible material.

In some implementations, the compartments have equal volume.

In some implementations, the wall portions of the partitioning device are solid.

In some implementations, the partitioning device is integrally formed with at least one side surface and/or bottom surface of the well.

In some implementations, the wall portions of the partitioning device prevent flow of a pourable culture substrate between the compartments. In some implementations, the wall portions of the partitioning device comprise one or more openings that allow for the flow therethrough of an uptake substance between the compartments. In some implementations, the wall portions of the partitioning device comprise a membrane that allow for the flow therethrough of an uptake substance between the compartments.

In some implementations, the partitioning device is created using additive manufacturing.

Various other implementations include a method for creating a competitive assay system. The method includes (1) inserting a partitioning device into a well in a culture well plate. The well is defined by a bottom surface through which a central axis extends and at least one side surface extending axially from the bottom surface.

The partitioning device includes a first wall, a second wall, and a third wall. The first wall portion has an upper edge, a lower edge, a first side edge, a second side edge, and first and second divider surfaces. The upper and lower edges are opposite and spaced apart from each other, the first and second side edges are opposite and spaced apart from each other, and the first and second divider surfaces are opposite and spaced apart from each other and extend between the upper and lower edges and the first and second side surfaces.

The second wall portion has an upper edge, a lower edge, a first side edge, a second side edge, and first and second divider surfaces. The upper and lower edges of the second wall portion are opposite and spaced apart from each other, the first and second side edges of the second wall portion are opposite and spaced apart from each other, and the first and second divider surfaces of the second wall portion are opposite and spaced apart from each other and extend between the upper and lower edges and the first and second side surfaces of the second wall portion.

The third wall portion has an upper edge, a lower edge, a first side edge, a second side edge, and first and second divider surfaces. The upper and lower edges of the third wall portion are opposite and spaced apart from each other, the first and second side edges of the third wall portion are opposite and spaced apart from each other, and the first and second divider surfaces of the third wall portion are opposite and spaced apart from each other and extend between the upper and lower edges and the first and second side surfaces of the third wall portion.

The lower edge of the first, second, and third wall portions are disposed against the bottom surface of the well. The first side edges of the first, second, and third wall portions are disposed in contact with at least one side surface of the well. The first and second divider surfaces of the first, second, and third wall portions define first, second, and third compartments in the well.

The method further includes (2) inserting a first pourable culture substrate including a first culture within the first compartment, (3) inserting a second pourable culture substrate including a second culture within the second compartment, wherein the first culture is different than the second cell culture, (4) inserting a third pourable culture substrate comprising a third culture within the third compartment, wherein the first and second cultures are different than the third culture, (5) removing the partitioning device from the well after the pourable culture substrates have solidified, and (6) introducing an uptake substance inside the well such that the uptake substance contacts each of the solidified culture substrate.

In some implementations, the second side edges of the first, second, and third wall portions intersect each other.

In some implementations, the first side edges of the first, second, and third wall portions are coupled to each other by a coupling wall, and the coupling wall is disposable against at least one side surface of the well.

In some implementations, the partitioning device further includes a center wall having a perimeter forming a closed shape, wherein the center wall is sealingly disposable between the second side edges of the first, second, and third wall portions.

In some implementations, the partitioning device is created using additive manufacturing.

In some implementations, the first, second, and third cultures comprise cell cultures. In some implementations, the first, second, and third cultures comprise bacterial cultures.

In some implementations, the first, second, and third pourable culture substrates comprise hydrogels.

Various other implementations include a method for creating a competitive assay system. The method includes (1) providing a partitioning device in a well in a culture well plate. The well is defined by a bottom surface through which a central axis extends and at least one side surface extending axially from the bottom surface.

The partitioning device includes a first wall, a second wall, and a third wall. The first wall portion has an upper edge, a lower edge, a first side edge, a second side edge, and first and second divider surfaces. The upper and lower edges are opposite and spaced apart from each other, the first and second side edges are opposite and spaced apart from each other, and the first and second divider surfaces are opposite and spaced apart from each other and extend between the upper and lower edges and the first and second side surfaces.

The second wall portion has an upper edge, a lower edge, a first side edge, a second side edge, and first and second divider surfaces. The upper and lower edges of the second wall portion are opposite and spaced apart from each other, the first and second side edges of the second wall portion are opposite and spaced apart from each other, and the first and second divider surfaces of the second wall portion are opposite and spaced apart from each other and extend between the upper and lower edges and the first and second side surfaces of the second wall portion.

The third wall portion has an upper edge, a lower edge, a first side edge, a second side edge, and first and second divider surfaces. The upper and lower edges of the third wall portion are opposite and spaced apart from each other, the first and second side edges of the third wall portion are opposite and spaced apart from each other, and the first and second divider surfaces of the third wall portion are opposite and spaced apart from each other and extend between the upper and lower edges and the first and second side surfaces of the third wall portion.

The lower edge of the first, second, and third wall portions are disposed against the bottom surface of the well, and the first side edges of the first, second, and third wall portions are disposed in contact with at least one side surface of the well. The first and second divider surfaces of the first, second, and third wall portions define first, second, and third compartments in the well.

The method further includes (2) inserting a first pourable culture substrate comprising a first culture within the first compartment, (3) inserting a second pourable culture substrate comprising a second culture within the second compartment, wherein the first culture is different than the second culture, (4) inserting a third pourable culture substrate comprising a third culture within the third compartment, wherein the first and second cultures are different than the third culture, and (5) introducing an uptake substance inside the well such that the uptake substance flows through the partitioning device and contacts each of the solidified culture substrates.

In some implementations, the second side edges of the first, second, and third wall portions intersect each other.

In some implementations, the first side edges of the first, second, and third wall portions are coupled to each other by a coupling wall, and the coupling wall is disposable against at least one side surface of the well.

In some implementations, the partitioning device further includes a center wall having a perimeter forming a closed shape, and the center wall is sealingly disposable between the second side edges of the first, second, and third wall portions.

In some implementations, the partitioning device is created using additive manufacturing.

In some implementations, the first, second, and third cultures comprise cell cultures. In some implementations, the first, second, and third cultures comprise bacterial cultures.

In some implementations, the first, second, and third pourable culture substrates comprise hydrogels.

### BRIEF DESCRIPTION OF DRAWINGS

Example features and implementations are disclosed in the accompanying drawings. However, the present disclosure is not limited to the precise arrangements and instrumentalities shown.

FIG. 1 is a perspective view of a competitive assay platform in accordance with one implementation.

FIG. 2 is a perspective view of a competitive assay platform in accordance with another implementation.

FIG. 3 is an example flow chart of a method of using the competitive assay platform of FIG. 1 according to one implementation.

FIG. 4 is a perspective view of a competitive assay platform in accordance with another implementation.

### DETAILED DESCRIPTION

The following is a description of various implementations of a competitive assay platform to evaluate selective uptake of substances in three dimensional cultures, such as cell cultures, bacterial cultures, etc.

Certain terminology is used herein for convenience only and is not to be taken as a limitation on the present claims. In the drawings, the same reference numbers are employed for designating the same elements throughout the several figures. A number of examples are provided, nevertheless, it will be understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

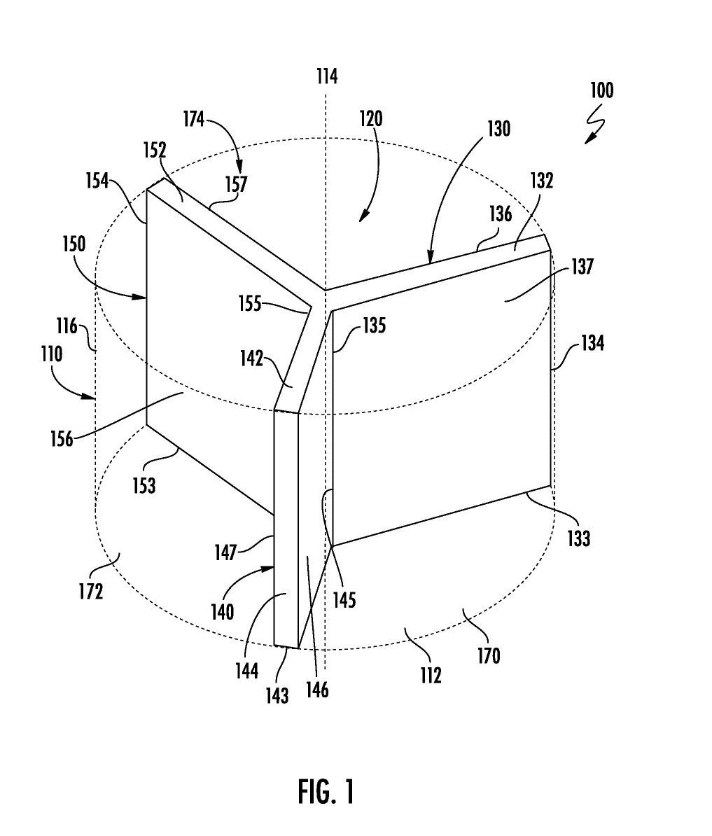

FIG. 1 shows one implementation of the competitive assay platform 100 with a partitioning device 120 disposed in a well 110. The well 110 has a bottom surface 112, a central axis 114, and a side surface 116. The bottom surface 112 and the side surface 116 define a well volume. The central axis 114 of the well 110 extends through the bottom surface 112 of the well 110, and the side surface 116 extends axially from the bottom surface 112. An edge of the side surface 116 opposite the bottom surface 112 defines an opening into the well 110. While the well 110 in FIG. 1 is cylindrically shaped and has a single side surface 116, a well having another prismatic shape and having more than one side surface could also be used. It is also understood that a well can be any study apparatus that can be utilized for studying competition, including cell culture well plates, petri dishes, etc.

The partitioning device 120 includes a first wall portion 130, a second wall portion 140, and a third wall portion 150. Each of the first 130, second 140, and third wall portions 150 has an upper edge 132, 142, 152, a lower edge 133, 143, 153, a first side edge 134, 144, 154, a second side edge 135, 145, 155, a first divider surface 136, 146, 156, and a second divider surface 137, 147, 157, respectively. The upper 132, 142, 152 and lower edges 133, 143, 153, respectively, are opposite and spaced apart from each other, the first 134, 144, 154 and second side edges 135, 145, 155, respectively, are opposite and spaced apart from each other, and the first 136, 146, 156 and second divider surfaces 137, 147, 157, respectively, are opposite and spaced apart from each other and extend between the upper 132, 142, 152, the lower edges 133, 143, 153, and the first 134, 144, 154 and second side edges 135, 145, 155, respectively. The second side edges 135, 145, 155 of each of the first 130, second 140, and third wall portions 150, respectively, intersect each other. FIG. 1 shows the wall portions 130, 140, 150 being integrally formed with each other, but it is understood that the wall portions 130, 140, 150 could also be separately formed and coupled together.

The partitioning device 120 of FIG. 1 is shown inserted into the well 110 with the lower edge 133 of the first wall portion 130, the lower edge 143 of the second wall portion 140, and the lower edge 153 of the third wall portion 150 of the partitioning device 120 disposed on (e.g., abutting) the bottom surface 112 of the well 110. The first side edge 134 of the first wall portion 130, the first side edge 144 of the second wall portion 140, and the first side edge 154 of the third wall portion 150 are disposed against (e.g., abutting) the side surface 116 of the well 110. Thus, when the partitioning device 120 is inserted into the well 110, the second divider surface 137 of the first wall portion 130 and the first divider surface 146 of the second wall portion 140 define a first compartment 170. The second divider surface 147 of the second wall portion 140 and the first divider surface 156 of the third wall portion 150 define a second compartment 172. And, the first divider surface 136 of the first wall portion 130 and the second divider surface 157 of the third wall portion 150 define a third compartment 174. The wall portions 130, 140, 150 of FIG. 1 are designed such that the first 170, second 172, and third compartments 174 have equal volumes. The first 130, second 140, and third wall portions 150 shown in FIG. 1 are planar, but the wall portions in other implementations of the partitioning device can be any other shape.

The wall portions 130, 140, 150 of the partitioning device 120 can be made from a biocompatible material such as a polymer (e.g., polylactic acid, polycaprolactone). However, the wall portions 130, 140, 150 can also be made from any non-biocompatible material.

The partitioning device 120 can be created using additive manufacturing, or any other means known in the art. The partitioning device 120 of FIG. 1 is solid and separately formed from the side surface 116 and bottom surface 112 of the well 110 so that the partitioning device 120 can be inserted into the well 110, and later removed from the well 110, as described below, before an uptake substance is introduced into the well 110. However, the partitioning device 120 may be integrally formed with the side surface(s) 116 and/or bottom surface 112 of the well 110 in some implementations. And, in some implementations, the wall portions can define holes or include a membrane. The holes or membrane are sized such that the culture substrate does not flow outside the respective compartment, but the uptake material inserted in the compartments is able to flow freely among each of the compartments through the holes or membrane.

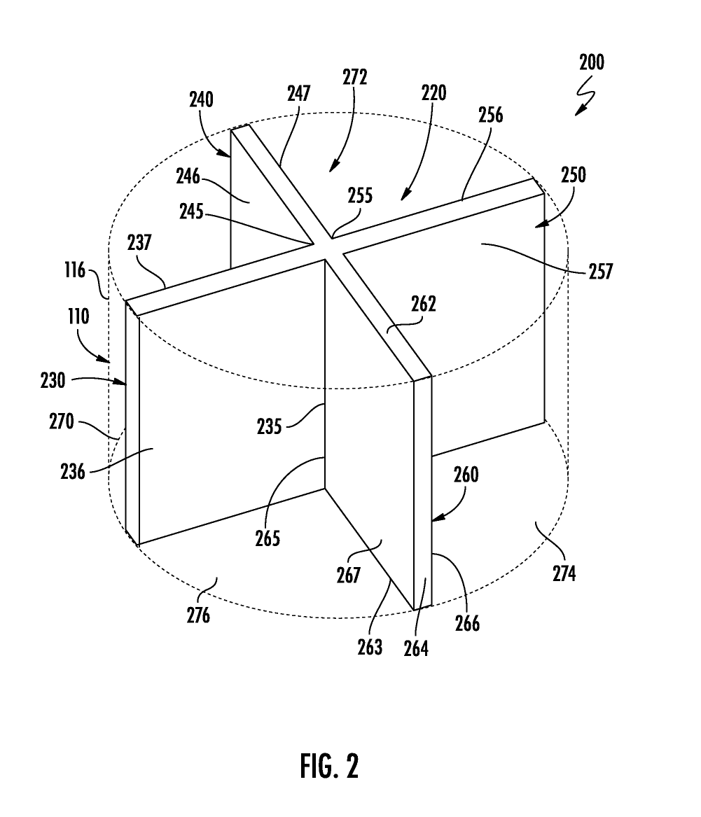

The partitioning device 120 shown in FIG. 1 has three wall portions 130, 140, 150. However, in other implementations, the partitioning device 120 can have more wall portions to form additional compartments. FIG. 2 depicts another implementation of a competitive assay platform 200 with a partitioning device 220 similar to the partitioning device 120 shown in FIG. 1, but the partitioning device 220 of FIG. 2 comprises a fourth wall portion 260. Similar to the other wall portions 230, 240, 250, the fourth wall portion 260 has an upper edge 262, a lower edge 263, a first side edge 264, a second side edge 265, a first divider surface 266, and a second divider surface 267. The second side edge 265 of the fourth wall portion 260 intersects the second side edges 235, 245, 255 of the first 230, second 240, and third wall portions 250, respectively. The first side edge 264 of the fourth wall portion 260 is disposed against the side surface 116 of the well 110. Thus, when the partitioning device 220 is inserted into the well 110, the second divider surface 237 of the first wall portion 230 and the first divider surface 246 of the second wall portion 240 define a first compartment 270. The second divider surface 247 of the second wall portion 240 and the first divider surface 256 of the third wall portion 250 define a second compartment 272. The second divider surface 257 of the third wall portion 250 and the first divider surface 266 of the fourth wall portion 260 define a third compartment 274. And, the second divider surface 267 of the fourth wall portion 260 and the first divider surface 236 of the first wall portion 230 define a fourth compartment 276.

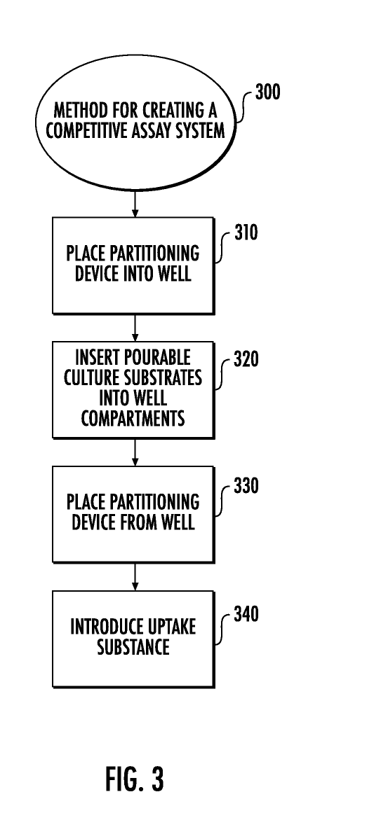

FIG. 3 shows the steps for an example method for creating a competitive assay system 300. In step 310, a partitioning device, such as partitioning devices 120, 220 described above in relation to FIGS. 1 and 2, is inserted into a well. The partitioning device is inserted into the well such that the bottom edges of the wall portions are disposed against the bottom surface of the well, and the first side edges of the wall portions are disposed against the side surface(s) of the well, and the second side edges of the wall portions intersect each other. In this way, the divider surfaces of the wall portions define multiple compartments in the well.

In step 320, different pourable culture substrates capable of setting as solids are inserted into each compartment, respectively, with each culture substrate and respective compartment containing a different culture. Pourable culture substrates can include any substance that can take a liquid form and subsequently solidify or partially solidify, for example, hydrogels, slurries, suspensions, solutions, etc. The culture substrates can contain various types of cultures, including cell cultures, bacterial cultures, etc.

Once the culture substrates have solidified, the partitioning device is removed from the well such that the culture substrates remain in place in the well, which is shown as step 330. In step 340, an uptake substance is introduced inside the well and is in contact with each of the solidified culture substrates.

Because each compartment has an equal volume and because equal volumes of culture substrates are inserted into each compartment, the culture substrates have equal surface areas for uptake of the uptake substance.

As mentioned above, in some implementations, the wall portions of the partitioning device can define holes or define a membrane, and the holes or membrane may be sized and/or selected such that the culture substrates inserted within each compartment are prevented from flowing outside each respective compartment, but an uptake material inserted in the compartments can flow freely between each of the compartments. In these implementations, the step of removing the partitioning device from the well can be eliminated and the partitioning device can be left in the well during the introduction of the uptake substance inside the well.

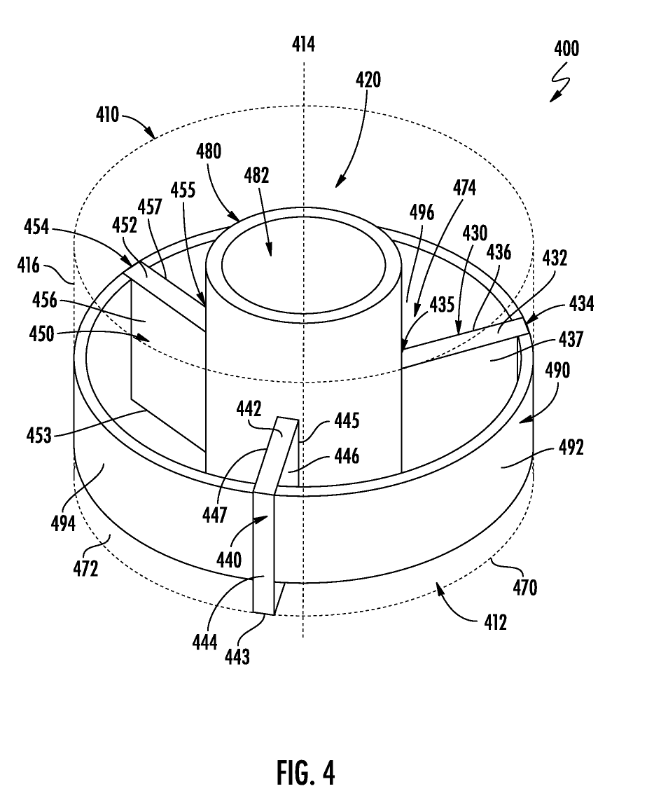

FIG. 4 depicts another implementation of a competitive assay platform 400 with a partitioning device 420 similar to the partitioning device 120 shown in FIG. 1, but the second side edges 435, 445, 455 of the first, second, and third wall portions 430, 440, 450 of the partitioning device 420 of FIG. 4 do not intersect each other. The partitioning device 420 shown in FIG. 4 also includes a coupling wall 490. The coupling wall 490 shown in FIG. 4 includes three coupling wall segments 492, 494, 496. Each of the three segments 492, 494, 496 of the coupling wall 490 couple a pair of adjacent first side edges 434, 444, 454 of the first, second, or third wall portions 430, 440, 450 to each other. Thus, a first segment 492 of the coupling wall 490 couples the first side edge 434 of the first wall portion 430 and the first side edge 444 of the second wall portion 440, a second segment 494 of the coupling wall 490 couples the first side edge 444 of the second wall portion 440 and the first side edge 454 of the third wall portion 450, and a third segment 496 of the coupling wall 490 couples the first side edge 456 of the third wall portion 450 and the first side edge 434 of the first wall portion 430. Each of the segments 492, 494, 496 of the coupling wall 490 are positioned such that the segments 492, 494, 496 of the coupling wall 490 are disposable against the side surface 416 of the well 410, which is shown in the dotted line of FIG. 4.

Although the coupling wall 490 shown in FIG. 4 includes multiple segments 492, 494, 496, in some implementations, the coupling wall includes one continuous wall coupled to the ends of the first side edges of the first, second, and third wall portions. In other implementations that include a partitioning device including more than three wall portions, the coupling wall includes any number of segments such that a segment couples the first edges of each set of adjacent wall portions. Although the segments 492, 494, 496 of the coupling wall 490 shown in FIG. 4 have an arcuate cross section as viewed in a plane parallel to the lower edges 433, 443, 453 of the first, second, and third wall portions 430, 440, 450, in other implementations, the coupling walls may have any other shape such that the first edges of each set of adjacent side walls are coupled and the partitioning device is disposable within the well.

The competitive assay platform 400 shown in FIG. 4 also includes an annular center wall 480 defining a center compartment 482. The annular center wall 480 is sized such that the annular center wall 480 is sealingly disposable between the second side edges 435, 445, 455 of the first, second, and third wall portions 430, 440, 450. Thus, a culture substrate or uptake substance introduced into the first compartment 470, the second compartment 472, the third compartment 474, or the center compartment 482 is prevented from flowing into any of the other compartments. The annular center wall 480 is separately formed from the second side edges 435, 445, 455 of the first, second, and third wall portions 430, 440, 450 such that the annular center wall 480 is removable from the well 410 separately from the remainder of the partitioning device 420. However, in other implementations, the annular center wall is integrally formed with the second side edges of the first, second, and third wall portions. Although the annular center wall 480 shown in FIG. 4 has a circular cross section as viewed in a plane parallel to the lower edges 433, 443, 453 of the first, second, and third wall portions 430, 440, 450, in other implementations, the cross section of the center wall as viewed in a plane parallel to the lower edges of the first, second, and third wall portions may have another shape, such as a triangle, a square, a pentagon, an oval, or any other closed shape.

In use, culture substrates are inserted into each of the first, second, and third compartments 470, 472, 474, as described above with respect to FIG. 3. An uptake substance is then inserted into the center compartment 482. Once the culture substrates have solidified, the annular center wall 480 is removed from the well 410 such that the uptake substance is introduced to the solidified culture substrates in each of the first, second, and third compartments 470, 472, 474. In this implementation, the remainder of the partitioning device 420 (the first, second, and third wall portions 430, 440, 450, and the coupling wall 490) can remain within the well 410.

### Example

A partitioning device, like the partitioning device 220 shown in FIG. 2 with four wall portions, was created using 3D printing. The partitioning device was manufactured using a biocompatible poly(lactic acid) (“PLA”) polymer which fits snuggly into a well of a standard cell culture 12 well plate. The partitioning device was used to create four competing compartments in 3D hydrogels (e.g., polyacrylamide hydrogels, collagen hydrogels), which subsequently permitted evaluation of selective nanoparticle uptake to test for competition. The compartments enabled incorporation of four different cell types, which were tested for selective nanoparticle uptake.

Four different cell types—(1) HepG2 liver cells, and breast cancer cells, (2) MDA-MB-231BR (a brain metastasizing variant of the triple negative breast cancer cell line MDA-MB-231), (3) MDA-MB-231, and (4) SKBr3—were seeded at 7,500 cells per gel compartment using Collagen-I hydrogels (5 mg/mL) in a single well of a 12 well plate. The cell-gel constructs were then incubated with 75 μg/mL of quinic acid-coated ultrasmall iron-oxide nanoparticles for three days followed by Prussian blue staining to evaluate cellular uptake.

The results of the uptake testing were as expected. Significant uptake of these nanoparticles was noted for HepG2 liver cells, followed by MDA-MB-231BR, and MDA-MB-231. No significant uptake was noted for SKBr3 cells. These results demonstrate the feasibility of the partitioning device disclosed herein.

A number of implementations have been described. Nevertheless, it will be understood that various modifications may be made without departing from the spirit and scope of the claims. Accordingly, other implementations are within the scope of the following claims.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A partitioning device for creating at least three separate three-dimensional cultures for studying selective uptake of a substance in the cultures, the partitioning device being disposable in a well, the well being defined by a bottom surface through which a central axis of the well extends and at least one side surface that extends axially from the bottom surface, the partitioning device comprising:
a first wall portion having an upper edge, a lower edge, a first side edge, a second side edge, and first and second divider surfaces, wherein the upper and lower edges are opposite and spaced apart from each other, the first and second side edges are opposite and spaced apart from each other, and the first and second divider surfaces are opposite and spaced apart from each other and extend between the upper and lower edges and the first and second side surfaces,
a second wall portion having an upper edge, a lower edge, a first side edge, a second side edge, and first and second divider surfaces, wherein the upper and lower edges of the second wall portion are opposite and spaced apart from each other, the first and second side edges of the second wall portion are opposite and spaced apart from each other, and the first and second divider surfaces of the second wall portion are opposite and spaced apart from each other and extend between the upper and lower edges and the first and second side surfaces of the second wall portion,
a third wall portion having an upper edge, a lower edge, a first side edge, a second side edge, and first and second divider surfaces, wherein the upper and lower edges of the third wall portion are opposite and spaced apart from each other, the first and second side edges of the third wall portion are opposite and spaced apart from each other, and the first and second divider surfaces of the third wall portion are opposite and spaced apart from each other and extend between the upper and lower edges and the first and second side surfaces of the third wall portion,
wherein the lower edge of the first, second, and third wall portions are disposable on a bottom surface of a well, and the first side edges of the first, second, and third wall portions are disposable against at least one side surface of the well, and
wherein the first and second divider surfaces of the first, second, and third wall portions at least partially define first, second, and third compartments.

2. The partitioning device of claim 1, wherein the second side edges of the first, second, and third wall portions intersect each other.

3. The partitioning device of claim 1, wherein the first side edges of the first, second, and third wall portions are coupled to each other by a coupling wall, the coupling wall being disposable against at least one side surface of the well.

4. The partitioning device of claim 1, further comprising a center wall having a perimeter defining a closed shape, wherein the center wall is sealingly disposable between the second side edges of the first, second, and third wall portions.

5. The partitioning device of claim 1, wherein each of the first wall portion, the second wall portion, and the third wall portion are planar.

6. The partitioning device of claim 1, wherein the first wall portion, the second wall portion, and/or the third wall portion are integrally formed.

7. The partitioning device of claim 1, further comprising a fourth wall portion, the fourth wall portion having an upper edge, a lower edge, a first side edge, a second side edge, and first and second divider surfaces, wherein the upper and lower edges of the fourth wall portion are opposite and spaced apart from each other, the first and second side edges of the fourth wall portion are opposite and spaced apart from each other, and the first and second divider surfaces of the fourth wall portion are opposite and spaced apart from each other and extend between the upper and lower edges and the first and second side surfaces of the fourth wall portion, wherein the first side edge of the fourth wall portion is disposable against at least one side surface of the well, and wherein first and second divider surfaces of the first, second, third, and fourth wall portions at least partially define the first, second, and third compartments and a fourth compartment.

8. The partitioning device of claim 1, wherein the partitioning device comprises a biocompatible material.

9. The partitioning device of claim 8, wherein the biocompatible material comprises a polymer.

10. The partitioning device of claim 9, wherein the biocompatible material comprises polylactic acid.

11. The partitioning device of claim 9, wherein the biocompatible material comprises polycaprolactone.

12. The partitioning device of claim 1, wherein the partitioning device comprises a non-biocompatible material.

13. The partitioning device of claim 1, wherein the compartments have equal volumes.

14. The partitioning device of claim 1, wherein the wall portions of the partitioning device are solid.

15. The partitioning device of claim 1, wherein the wall portions of the partitioning device prevent flow of a pourable culture substrate between the compartments.

16. The partitioning device of claim 15, wherein the wall portions of the partitioning device comprise one or more openings through which uptake materials flow between the compartments.

17. The partitioning device of claim 15, wherein the wall portions of the partitioning device comprise a membrane through which uptake materials flow between the first and second compartments.

18. The partitioning device of claim 1, wherein the partitioning device is created using additive manufacturing.

19. The partitioning device of claim 1, further including at least one well, the at least one well defined by a bottom surface through which a central axis extends and at least one side surface extending axially from the bottom surface, wherein the first, second, and third wall portions are disposed within the well.

20. The partitioning device of claim 19, wherein the lower edges of the first, second, and third wall portions are disposed against the bottom surface of the well and the first side edges of the first, second, and third wall portions are disposed in contact with at least one side surface of the well.

21.-55. (canceled)
